(19) United States
(12) Patent Application Publication
(10) Pub. No.: US 2006/0116556 A1
(43) Pub. Date: Jun. 1, 2006

(54) Physiological assessment system and method

(76) Inventor: Paul Duhamel, Val Caron (CA)

Correspondence Address: Paul Duhamel, P.O. Box 106, Val Caron, P3N 1N7, CA

(21) Appl. No.: 10/999,167

(22) Filed: Nov. 29, 2004

(51) Int. Cl. A61B 5/00

(52) U.S. Cl. 600/300

(57) ABSTRACT

A system and method for physiological assessment utilizes electrophysiological techniques, such as evoked potential (EP) data. Symptomatic and asymptomatic sensory areas are stimulated and EP data are collected. Artifacts can optionally be deleted from the raw EP data, which can then be appropriately filtered and transformed using software filtering and signal processing transformation techniques. Points of interest are detected and labeled on the resulting traces, which are output for analysis, diagnosis and treatment purposes. Differences between symptomatic and asymptomatic EPs provide objective, quantitative, repeatable information about pathological conditions.

This application claims the benefit of U.S. Provisional Patent Application Ser. No. 60/464,2003 filed Apr. 22, 2003, the contents of which are hereby expressly incorporated by reference in their entirety.

### BACKGROUND OF THE INVENTION

1. Field of the Invention

The present invention relates generally to physiological assessment systems and methodologies, and in particular to a system and method for detecting, recording, analyzing, evaluating, and diagnosing pathological sensory and perceptual phenomena, such as pain, using evoked potentials (EPs).

2. Description of the Related Art

Effective treatment and prevention of medical conditions often depends on objective and accurate measurement of the underlying pathological processes. However, many of the symptoms for which patients commonly seek treatment are difficult to measure objectively. For example, the objective assessment of pain has presented difficulties, particularly in view of the highly subjective nature of the experience of pain. The most widely used method of measuring pain involves having patients describe their symptoms or having them rate their symptoms using Likert-type scales (e.g., “On a scale of 1 to 10, how intense is your pain presently.”). Although self-reports are undoubtedly an important source of information concerning the nature of patients' symptoms, a substantial body of evidence suggests that self-reports provide data about patients' symptoms that is neither reliable nor valid in many circumstances. Such self-reports are used by health care insurance providers and other organizations for authorizing and reimbursing for medical care, such as extended hospital stays. However, the subjective nature of such reports, and the current lack of an effective method to verify same, tend to reduce their reliability and usefulness.

The problems that currently exist in the measurement of pain come at a significant cost to people suffering from pain, medical professionals responsible for treating pain, and society in general. Patients who have pain in the absence of any known organic etiology are sometimes suspected of malingering. Consequently, they may be denied potentially helpful treatments and may also be denied compensation in situations where pain is the result of someone else's negligence. For health professionals treating pain, the problems associated with the measurement of pain have numerous ethical and legal implications. For example, incorrectly diagnosing the intensity and chronicity of pain because of inaccurate or incomplete self-reports could result in patients receiving expensive, unnecessary and potentially harmful treatments (e.g., surgery, narcotics, etc.). When serious complications occur as a result of treatment, the evidence justifying treatment is subjective. The cost to society is also substantial. Pain is the most common symptom for which patients seek medical treatment.

Researchers have employed a variety of different technologies in attempting to measure pain intensity and all have their particular strengths and weakness. For example, several endogenous substances such as histamine and prostaglandin E2 have been found to correlate with self-reported pain intensity and are readily measured with biochemistry. These biochemical measures, however, are sometimes invalid since these substances are often present in asymptomatic tissue (i.e., tissue which is not painful). Imaging technologies such as magnetic resonance imaging and positron emission tomography have been shown to validly and reliably measure the intensity of experimentally induced phasic pain and its functional anatomy. Imaging technologies, however, have not been successful in measuring clinical pain or tonic pain. Electrophysiological measures, such as electromyography and background electroencephalography, have also been employed but have not been successful as of yet. One preferred embodiment of the current invention involves measuring pain intensity using a type of electrophysiological measure called an evoked potential (EP). Our research has shown that certain characteristics of EPs vary systematically with pain intensity. EPs have been studied successfully in connection with many pathological sensory and perceptual processes such as color vision and tone perception. EPs can be detected and processed using electroencephalogram (EEG) equipment and methodologies, such as those described and shown in Pichimayr et al. U.S. Pat. No. 5,846,208, and Schultz et al. U.S. Pat. No. 6,011,990, the disclosures of which are incorporated herein by reference. The advantages of using EEG equipment for EP detection and analysis include the non-invasive nature of such procedures, the commercial availability of BEG equipment and a high degree of reliability and repeatability when used to implement the system and method of the present invention.

Heretofore there has not been available a system and method for assessing physiological conditions with the advantages and features of the present invention. These advantages and features include, but are not limited to, developing an objective and practical measure of clinical musculoskeletal pain.

### BRIEF DESCRIPTION OF THE INVENTION

In the practice of one aspect of the present invention, electrophysiological signals are collected and evaluated in connection with symptomatic and asymptomatic sensory areas, such as tissue.

For example, separate sensors can be placed to record electrophysiological data, such as evoked potentials elicited by stimulation of symptomatic and asymptomatic tissue. Patient-specific databases can be created for comparing electrophysiological data collected at different times in order to evaluate the onset of pathological conditions and the effectiveness of their treatment. The characteristics of the EP signals, such as latency and amplitude of peaks and troughs in the waveforms, can be significant in assessing physiological responses of individual subjects. Moreover, the location of recording electrodes placed on the body, such as on the surface of the scalp or on the skin above the spinal cord, is an important variable in practicing the method of the present invention because of the nature of signal transduction and transmission throughout the nervous system from the receptors to the cortex through the peripheral nerves and spinal cord and because of the way electrophysiological signals can be attenuated by soft tissue and bone and how the pathological process is distributed in the anatomy. Characteristics of the sensory and perceptual sequalea of the pathological processes are detectable in the EPs. Such characteristics can be stored in a database for comparison with comparable characteristics obtained during other time periods. Such comparisons provide useful data for use in connection with treatment programs, diagnosis and assessment.

The reliability and validity of the data recovered can be enhanced with suitable mathematical transformations, using various filters and signal processing techniques.

The data collected in this manner can be utilized in conjunction with individual patient treatment programs, and can also be collected and processed for creating mathematical and statistical models for entire populations and demographic subgroups. The measurable, repeatable and consistent qualities associated with the data tend to make them highly accurate and reliable for assessment, diagnosis and treatment purposes.

To Applicants' knowledge, Applicants are the first to provide an effective method for objectively measuring or confirming physiological conditions, such as pain. A difference in electrophysical measurements, such as a difference in the response amplitude of evoked potential measurements between symptomatic and asymptomatic areas, may indicate the presence or absence of pain. Providing effective confirmation of pain can be beneficial for many industries, including the medical and insurance industries.

For example, the insurance industry may benefit from being able to objectively measure whether a claimant is experiencing pain or is malingering. The legal field may use this technique to prove or disprove elements of damages, and possibly aid in standardizing damage awards.

Because of its classification as a vital sign, hospitals are now required to check all incoming patients for pain. It would be beneficial for hospitals to have an objective measurement in place of, or in addition to, subjective reports of pain. In other embodiments, the present invention may be used to determine when a patient receives pain medication. The present invention may be used to test the effectiveness of pain medications.

Not all individuals have verbal capabilities, such as infants and those suffering from certain disabilities. The present invention provides a way to measure or confirm the presence or absence of pain in these individuals. The present invention may be used to establish the degree of a disability from which a person is suffering.

In a particularly preferred embodiment, objective electrophysiological measurements are compared to subjective measurements, such as self-report forms. Objective electrophysiological measurements may include data associated with an asymptomatic sensory area and data associated with a symptomatic sensory area. In certain embodiments, the objective and subjective data are acquired at substantially the same time, such as during the same experimental procedure. In a particularly preferred embodiment, the electrophysical measurements and the subjective measurements are normalized to a common scale and a correlation between electrophysical measurements and subjective data can be observed.

There are other alternative or preferred aspects of the present invention. They will become apparent as this specification proceeds. In this regard, this Brief Summary of Aspects of the Invention and Preferred Embodiments is not to be construed as itself limiting of the invention or various aspects of the invention or its preferred embodiments or as requiring that a given embodiment of the invention must include all features or advantages recited herein.

### BRIEF DESCRIPTION OF THE DRAWINGS

The drawings constitute a part of this specification and include exemplary embodiments of the present invention and illustrate various objects and features thereof.

FIG. 1 is substantially a block diagram of a system for physiological assessment embodying an aspect of the present invention.

FIG. 2 is substantially a flowchart of a physiological assessment method embodying an aspect of the invention.

FIG. 3 is a flowchart for evaluating evoked potentials (EPs) embodying an aspect of the invention.

FIG. 4 is a plot showing the distribution derived using a probability density density function for the EP data.

FIG. 5 is a chart showing EP signal traces over an evaluation period comparing symptomatic and asymptomatic responses.

FIG. 6 is a chart showing the EP signal traces for symptomatic and asymptomatic responses, with an area therebetween providing a quantitative measure of pathological condition intensity.

FIG. 7 is a chart showing wavelet transformed N4 (WN4r) amplitudes over the evaluation period comparing symptomatic and asymptomatic responses.

FIG. 8 is a chart showing wavelet transformed P5 (WP5r) latency over the evaluation period comparing symptomatic and asymptomatic responses.

FIG. 9 is a chart showing wavelet transformed P5 (WP5r) amplitudes over the evaluation period comparing symptomatic and asymptomatic responses.

FIG. 10 shows McNemar test results comparing the Day 2 to Day 3 trends of self-reported pain intensity, WN4r amplitude, WP5r latency and PGE2.

FIG. 11 shows binomial test results for correct vs. incorrect classification of symptomatology based on wavelet transformed relative N4 (WN4r) amplitude.

### DETAILED DESCRIPTION OF THE INVENTION

I. Introduction

As required, detailed embodiments and/or aspects of the present invention are disclosed herein; however, it is to be understood that the disclosed embodiments/aspects are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

II. EP Physiological Assessment System and Method

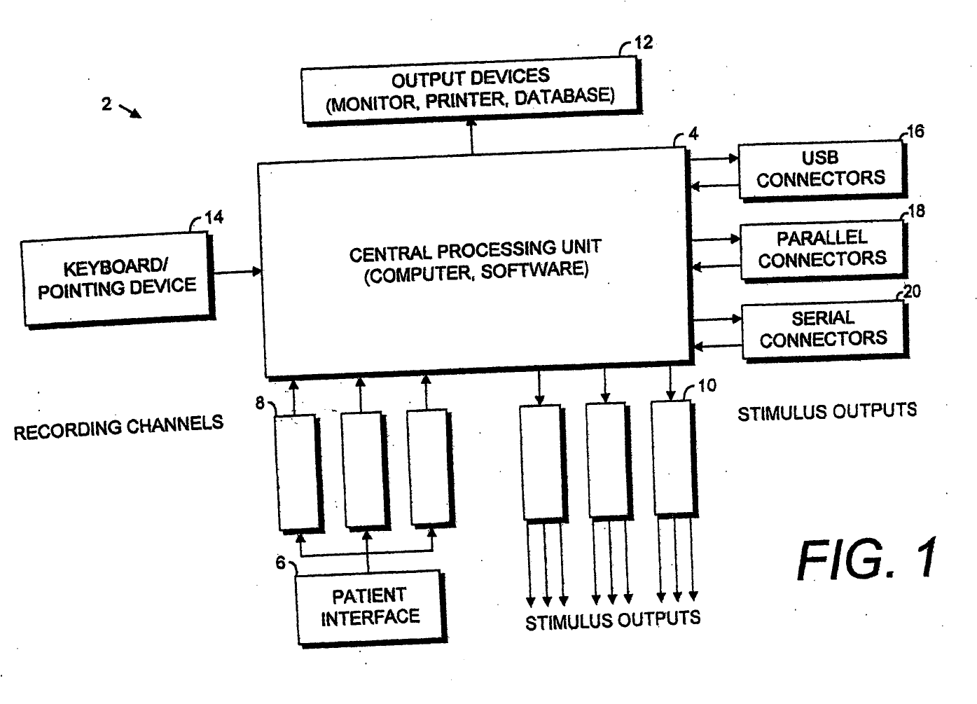

Referring to the drawings in more detail, the reference numeral 2 generally designates a system for analyzing EPs embodying an aspect of the present invention. As shown in FIG. 1, the system 2 generally comprises a central processing unit (CPU) with a source device, which can comprise a suitable personal computer, programmable logic controller (PLC) or other suitable programmable control device. A patient interface 6 can comprise an EEG device, or some other suitable patient interface adapted to receive electrical or magnetic signals and impulses from the patient. One or more recording channels (biopotential amplifiers) 8 are connected to the CPU 4 for transferring data thereto and receive electrophysiological activity signals from the patient interface 6. The recording channels 8 and the stimulation units 10 can be attached as peripherals or as modules that plug directly into the device. The recording channels 8 can be attached to patients either directly via leads 9 exiting the recording channels 8, or via a multi-channel patient interface 6. One or more stimulation units 10, such as electrodes, are connected to the CPU 4 and provide stimulus outputs 12 to a subject. Additional recording channels 8 and stimulation units 10 can be provided as modular units for attachment and detachment as needed.

An input device such as a keyboard or a pointing device (e.g., a mouse) 14 is operably connected to the CPU 4. Various output devices can be utilized with the system 2 of the present invention, and receive signals from the CPU 4, as shown in FIG. 1. For example, monitors, printers, databases, a network or the world wide web (“Internet”) can all receive output from the CPU 4. Such output can represent electrophysiological signals for recording and display. The CPU 4 can be provided with various suitable connectors and ports, such as the USB connector 16, parallel connectors 18 and serial connectors 20. Two-way communication and signal exchange are accommodated between the CPU 4 and the connectors 16, 18, 20. For example, the connectors can be used for other peripherals, I/O devices, a network connection or a connection to the world wide web (“Internet”).

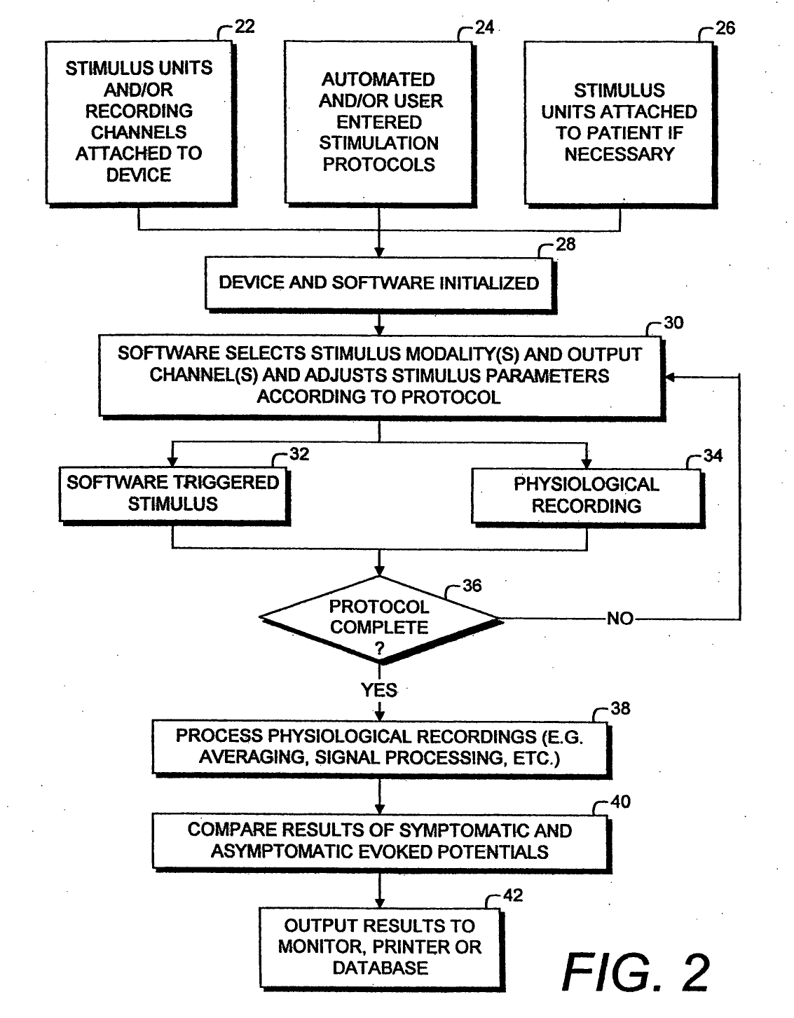

FIG. 2 shows a method for physiological assessment embodying one aspect of the invention. Stimulus units and/or recording channels are attached to or removed from the device as needed at 22. Automated and/or user programmed stimulation protocols can be selected via software at 24. The system's interface with the patient, such as attaching stimulus units, is prepared at 26. The software and the system are initialized at 28. The software selects and controls the stimulus modality(s) and output channel(s), and adjusts the stimulus parameters according to the chosen protocol at 30. Stimulus is triggered by the software at 32 and physiological reactions are recorded at 34. The delay between the stimulus onset and the electrophysiological recording can be manually selected or determined automatically by the software.

From the “Protocol Complete?” decision box 36, the stimulus selection, stimulus trigger and physiological recording steps 30-34 repeat if the protocol is not complete (negative branch from 36). An affirmative decision at 36 leads to averaging and signal processing functions of the physiological recordings at 38 to filter noise out of the raw recordings and to produce EP traces. The software then compares the EPs elicited by stimulation of the patient's symptomatic and asymptomatic sensory systems at 40. The results are output to an output device (as described above) at 42. As used in this application, “symptomatic” refers to aspects of a patient that have a condition or are indicated as having a condition and “asymptomatic” refers to aspects of the patent that do not have the condition or are not indicated as having the condition.

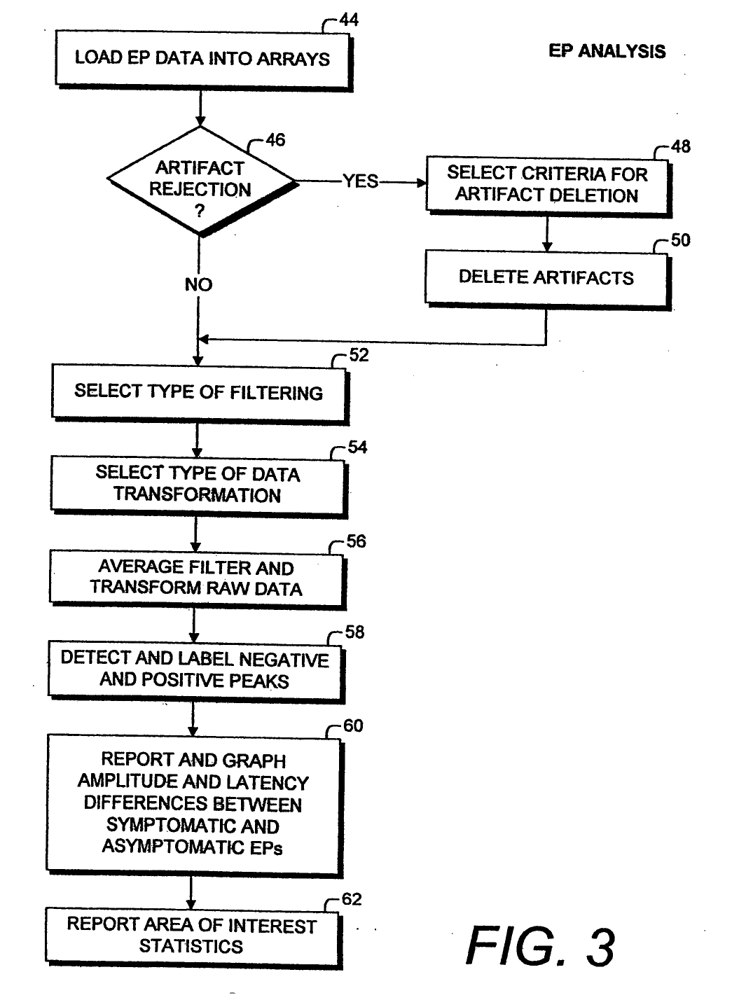

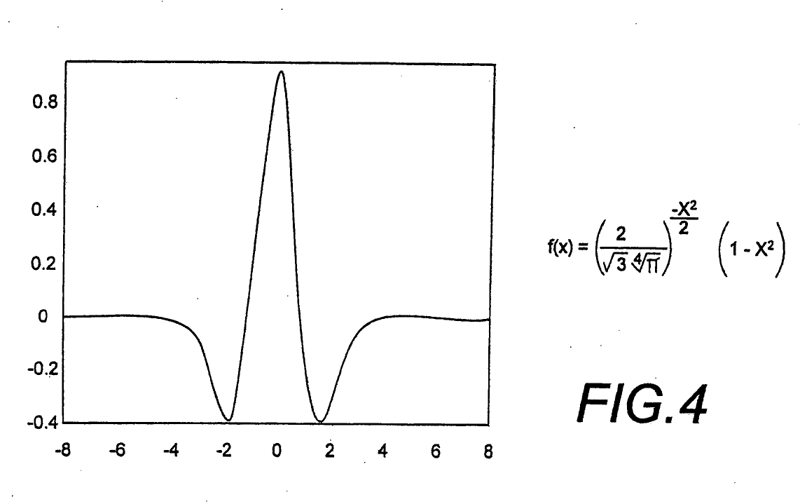

FIG. 3 shows the EP analysis step 38 in more detail. EP data is loaded into arrays at 44. At decision box 46 users can select if artifact rejection should occur and, if affirmative, select the criteria for artifact deletion at 48. Artifacts are then deleted at 50 and the method returns to a select type of filtering step at 52 (e.g., Butterworth, Chebyshev, Hamming Window, etc.). Type of data transformation is selected at 54. Examples included wavelet and spectrogram. A type of wavelet transformation using a second derivative of a Gaussian probability density function is shown in FIG. 4. Applying certain parameters to the wavelet analysis improves the accuracy of automated identification of certain EP components and the reliability and validity of the method. The raw data is averaged, filtered and transformed at 56 and the negative and positive peaks are detected and labeled at 58. By way of example, the software can be written to detect the first peak and valley in the wavelet transformed EP (wEP) occurring at approximately 100 ms after stimulus onset. The amplitudes and latencies of the first peak and valley are automatically recorded by the software and stored on the CPU memory device (e.g., hard drive).

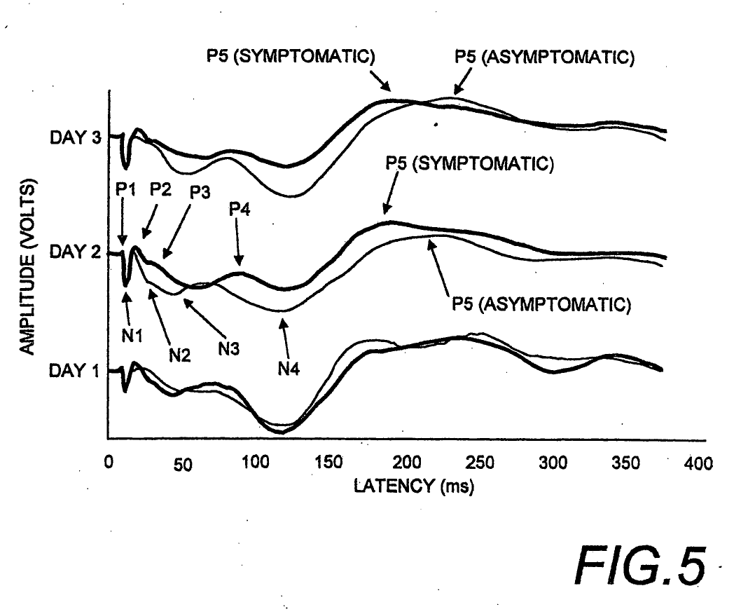

FIG. 5 shows the results of cortical EPs elicited by stimulating symptomatic and asymptomatic sensory areas of a subject over three days. The validation procedure involved experimentally induced pain in the form of delayed onset muscle soreness (DOMS), although the invention is not limited to such applications. Without limitation on the wide range of useful applications of the present invention, an exemplary application involves analyzing pathological conditions, such as pain. The methodology is verified by stimulating both symptomatic and asymptomatic tissue, and comparing the responses in the form of EP traces. For example, the comparison can be made between different body parts, such as a symptomatic bicep and an asymptomatic bicep. Moreover, asymptomatic responses can be recorded for comparison at a later time as symptomatic situations arise. For example, pre-surgery (asymptomatic) data can be recorded for comparison to post-surgical (symptomatic) data in order to monitor the efficacy of pain management procedures and pharmacologicals in surgical patients.

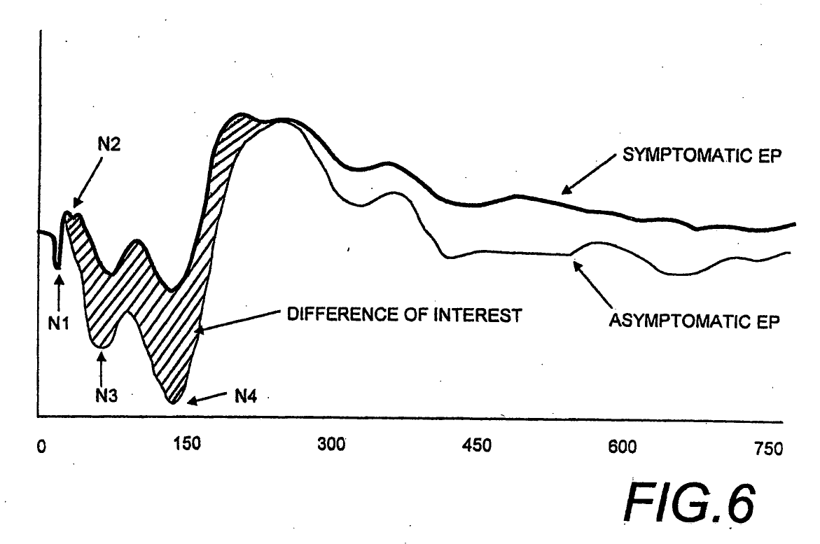

Referring to FIG. 5, deflections in the EP traces are labeled according to their directions, i.e. “P” for a positive or upward deflection and “N” for a negative or downward deflection. The numeral designations indicate their positions in the trace. Thus, P1 identifies the first positive deflection in the EP trace, and N2 identifies the second negative deflection. As shown in FIG. 5, amplitude and latency differences exist between symptomatic and asymptomatic EP components. Such differences are particularly pronounced on traces for Day 3. FIG. 6 shows the EP traces resulting from electrical stimulation of painful (symptomatic) and pain-free (asymptomatic) biceps in a subject. The area between the traces labeled “Difference of Interest” represents the intensity of pain reported in the painful tissue. The onset time and amplitude valence of the EP corresponding to the sensation of pain intensity is dependant on the involved anatomy. For example, in the case of painful biceps, the onset of the pain response occurs at approximately 150 ms and is on a negative component.

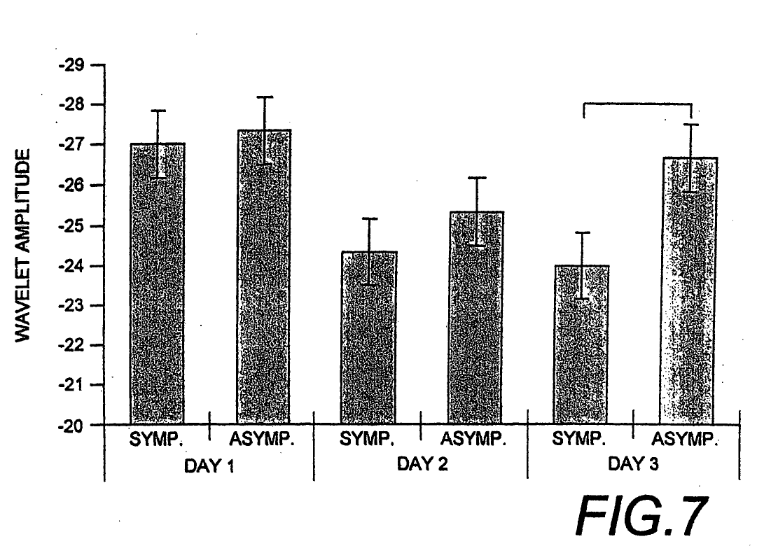

FIG. 7 shows the average symptomatic and asymptomatic wavelet transformed N4 (WN4r) amplitudes on three consecutive days. On Day 3, symptomatic WN4r amplitude was significantly smaller than asymptomatic WN4r amplitude (t(1, 11)=5.017, p<0.001). The differences on Day 1 and Day 2 are not statistically significant. The trend is therefore toward smaller WN4r amplitudes from Day 1 to Day 3.

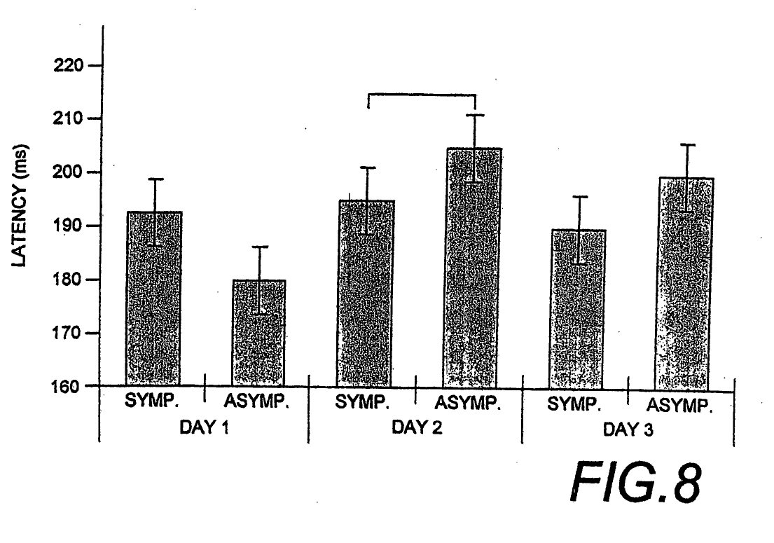

FIG. 8 shows the average wavelet transformed symptomatic and asymptomatic P5 (WA5) latencies from Day 1 to Day 3. On Day 2, symptomatic WP5 latency was significantly shorter than asymptomatic WP5 latency (t(1, 11)=−2.548, p=0.027). Day 1 and Day 3 differences are not statistically significant.

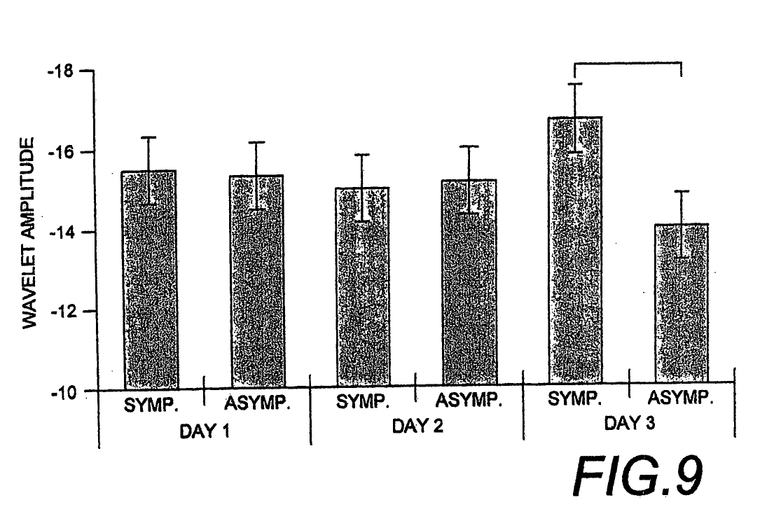

FIG. 9 shows the average wavelet transformed P5 (WP5) amplitudes from Day 1 to Day 3. On Day 3, symptomatic WP5 latency was significantly longer than asymptomatic WP5 latency (t(1, 11)=−2.228, p=0.048).

As stated, one of the utilities of the present invention is to record objective, physiological measures of pathological sensory and perceptual conditions. Previously, it has not been possible to correlate the results of objective techniques to results from commonly used subjective techniques, such as subjective self-reports. In order to be validated, objective techniques should be comparable to subjective techniques. Such analysis verifies that differences in electrophysical measurements between asymptomatic sensory areas and symptomatic sensory areas provide an objective measurement or confirmation of the presence or absence of a physiological condition, such as pain. For example, using the evoked potential technique, a difference in response amplitudes of asymptomatic and symptomatic sensory areas has been verified as indicating the presence of pain.

Heretofore, direct correlational analysis between the physiological measure and the subjective self-report has not been very meaningful because of the subjectiveness inherent in the raw data of these techniques. In fact, for the current example, no correlations were found when using the raw data. Having a patient choose a number on a Lickert scale that best represents his/her pain intensity is inherently a subjective task. The fact that the person may be required to choose a representative number on several occasions adds even more subjectivity to the measure. Therefore, using raw values (e.g. ‘4’ vs. ‘8’) may simply mean that one classification is ‘more painful’ than the other (not, in this example, that one is ‘twice’ as painful as the other).

For physiological measures, there are component characteristics that can reduce the usefulness of objective data. For example, response amplitudes may be susceptible to inconsistency due to extraneous variables. There are individual differences in amplitude measures that correlate to some degree with age, gender and race. Amplitudes from older people are attenuated because the skull thickens with age. Thickness of skull is also a property of gender and to some degree race.

Diet is another contributing factor. Sodium in a person's diet will determine the amount of electrolytes in the skin, which directly affects the conductance of an electrophysiological signal. Placement of the electrodes on the scalp will also contribute to amplitude differences because moving the electrode by only a few millimeters will reposition it in respect to the locus of the response.

Comparing symptomatic and asymptomatic response amplitudes across days may allow for extraneous variables such as diet and electrode placement to impact physiological measurements. For example, a doubling of a particular amplitude may not necessarily correspond to a doubling of the sensation felt by a person. It may simply be that the person had a salty breakfast that morning. It may be that this data simply allows one to classify one measure as being ‘more painful’ than the other.

For these reasons, it is advantageous to convert subjective raw data to a more objective measurement system. In the current example of taking measures over a three day period, it is helpful to code responses from the three days according to the degree of differences from a baseline. For instance, self-report measures for the three days can be coded as 0, 1 or 2, with 0 representing the day with the least reported pain and 2 representing the day with the most reported pain. The remaining self-report measures may then be normalized to force a distribution between 0 and 2. In the same way, physiological and biochemical measures across days can be coded so that the day with the least evidence of pain receives a value of 0 and the day with the most evidence of pain receives a 2. Once the data is represented in this objective scale, comparisons and correlations between measurement types are more meaningful.

Physiological and subjective behavior subjected to this analysis showed excellent correlation. Days with higher normalized values for self-report of pain showed higher normalized values for physiological measurements. The accuracy of this method was verified statistically.

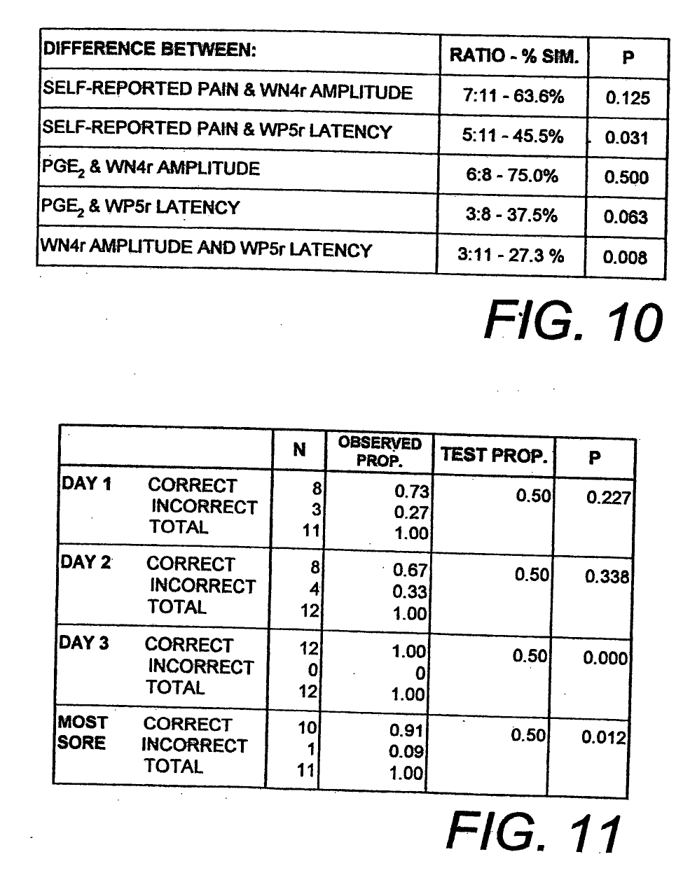

For example, FIG. 10 shows McNemar test results comparing the Day 2 to Day 3 trends of self-reported pain intensity, WN4r amplitude, WP5r latency and PGE2. Trends were determined by recoding the raw and averaged data from any two categories of comparative interests to binomial values of either 0 or 1, the number 0 referring to a similar trend. For instance, if self-reports indicated greater pain intensity on Day 3 compared to Day 2 and WN4r amplitude differences were greater on Day 3 compared to Day 2, both self-reports and WN4r amplitudes were assigned a value of 0, meaning there was a similar trend. If, on the other hand, WN4r amplitude differences were greater on Day 2 compared to Day 3, one measure was assigned a value of 0 and the other was assigned a value of 1 (e.g., self-reports=0, WN4r amplitudes=1), meaning there was not a similar trend. So in this example, the 1 would indicate that the self-report did not match or correlate with the physiological measurement of WN4r amplitude. This type of binomial analysis confirms the correlational technique.

The table in FIG. 10 shows pair-wise comparisons between the Day 2 to Day 3 trends of each variable. The probability levels less than 0.05 indicate a statistically significant difference between the trends of two variables. Self-reported pain, WN4r amplitudes and PGE2 are not significantly different, therefore they show similar trends. This is the first known evidence presented for a technique and method of analysis to correlate the subjective experience of pain intensity to an objective, physiological measurement technique. Because the current technique accurately measure, determine, or confirm conditions as described via self-reports, the current technique may find application in a number of areas. Some uses for, and advantages provided by, the present invention are discussed in more detail below.

FIG. 11 shows binomial test results for correct vs. incorrect classification of symptomatology based on wavelet transformed relative N4 (WN4r) amplitude. WN4r amplitudes were used to discriminate between symptomatic and asymptomatic sensory areas on Days 1, 2 and 3 and on the day the subjects reported the most intense delayed onset muscle soreness (“DOMS”) symptoms. WN4r amplitudes on Day 3 and on the day the subjects reported the most intense DOMS symptoms correctly classified symptomatic sensory areas 100 percent and 91 percent of the time. Classification rates on Days 1 and 2 did not reach statistical significance.

III. EP Analysis Applications

A wide range of useful applications exist for the system and method of the present invention. Thus, physiological conditions, including those associated with various pathologies, can be monitored and quantified. Treatment efficacies can be evaluated for purposes of prescribing optimum treatment protocols and pharmacologicals. Moreover, objective data can be gathered for assessing the intensity of pathological conditions, such as pain.

The field of pharmaceutical testing can benefit from the present invention. For example, considerable resources are devoted to developing, testing and perfecting pain medications. The objective data available from the system and method described above can enable researchers to verify the efficacy of new products and treatment procedures. Still further, the field of forensics can benefit from objective evidence and form of EP data, from which pathological conditions, or the absence thereof, can be proven with respect to individual subjects.

The field of health-care (i.e. medical and dental) benefits can also benefit from applications of the present invention. For example, treatment decisions that are presently made on the basis of subjective, self-evaluation type data can be based instead on objective data obtained with the physiological assessment system and method of the present invention. Such treatment decisions can involve significant costs to patients, health care providers and third parties with payment and reimbursement responsibilities. The objective data obtained through the analysis of EP responses according to the present invention can accurately determine levels of physiological response to such conditions as pain. Thus, the present invention can find application in connection with evaluating patients' conditions for purposes of prescribing appropriate treatments, assessing the need for hospitalization, determining the extent of patients' temporary and permanent disabilities and recording the effectiveness of physical therapy and other treatment programs.

The present invention may also find application in the provision of pain medication. For example, a patient could be periodically tested for pain and, if the patient is experiencing pain, medication could manually or automatically be dispensed to the patient.

The insurance industry may also benefit from having an objective test for the presence of pain. Because pain has been difficult to provide objectively, insurance companies often to do not know if claimants complaining of pain are in fact experiencing pain. The present invention provides a way to objectively test this by using electrophysiological tests to compare asymptomatic sensory areas to symptomatic sensory areas. If a difference in the electrophysical data is observed, such as a change in the response amplitude of evoked potential tests of symptomatic and asymptomatic sensory areas, then the test has confirmed the presence of pain. If an insignificant difference in response amplitude is observed, the test will indicate the absence of pain. This methodology may find similar use in the legal field in proving or disproving injury and pain and suffering, as well as providing a way to calibrate legal awards.

Hospitals have classified as pain as the fifth vital sign. In addition to vital signs such as heart rate and breathing, hospitals are now required to check for the presence of pain every time a patient is admitted. Currently, hospitals are forced to rely on subjective self-reports of pain. It would be beneficial to use the present method to confirm the presence or absence of pain.

Non-verbal humans, such as infants and those suffering from disabilities, may be unable to use subjective measures of pain. The present method may be used to confirm the presence or absence of pain in such individuals. For those suffering from a disability, the present invention may be used to help measure the degree of disability.

It is to be understood that while certain aspects and embodiments of the present invention have been disclosed herein, it is not limited thereto.

What is claimed is:

1. A electrophysiological assessment method of the type useable to assess a physiological condition, the electrophysiological assessment method comprising the steps of:
(A) collecting at least one electrophysiological signal of an asymptomatic sensory area to obtain asymptomatic response data;
(B) collecting at least one electrophysiological signal of an symptomatic sensory area to obtain symptomatic response data; and
(C) comparing the symptomatic response data to the asymptomatic response data to assess a physiological condition.

2. The method of claim 1, the step of comparing the asymptomatic response data to the symptomatic response data comprising analyzing a first data element comprising a latency.

3. The method of claim 2, the step of comparing the asymptomatic data to the symptomatic data further comprising analyzing a second data element associated with the first latency.

4. The method of claim 3, wherein the second data element comprises a response amplitude signal component.

5. The method of claim 1, the step of comparing the asymptomatic response data to the symptomatic response data comprising analyzing a first data element comprising a response amplitude signal component.

6. The method of claim 7, the step of comparing the asymptomatic response data to the symptomatic response data further comprising analyzing a second data element associated with the first data element.

7. The method of claim 1, the step of comparing the asymptomatic response data to the symptomatic response data comprising determining the difference between an amplitude of the asymptomatic response data at a first latency to an amplitude of the symptomatic response data at the first latency.

8. The method of claim 1, the step of comparing the asymptomatic response data to the symptomatic response data comprising determining at least one inflection point for the symptomatic response data and the asymptomatic response data and comparing the at least one inflection point of the asymptomatic response data to the at least one inflection point of the symptomatic response data.

9. The method of claim 1, the step of comparing the asymptomatic data to the symptomatic data comprising determining at least a first inflection point for the asymptomatic response data and at least a second inflection point for the symptomatic response data and comparing the amplitude and latency of the first and second inflections points.

10. The method of claim 1, the step of collecting at least one electrophysiological signal of the asymptomatic sensory area comprising stimulating the asymptomatic sensory area and recording the resulting physiological response.

11. The method of claim 1, the step of collecting the at least one electrophysiological signal of the symptomatic sensory area comprising stimulating the symptomatic sensory area and recording the resulting physiological response.

12. The method of claim 1, wherein the electrophysiological assessment method measures a somatosensory condition.

13. The method of claim 12, wherein the somatosensory condition comprises pain.

14. The method of claim 1, wherein the electrophysiological method is used to confirm the presence or absence of a somatosensory condition.

15. The method of claim 14, wherein the somatosensory condition comprises pain.

16. The method of claim 1, the step of collecting the at least one electrophysiological signal of an asymptomatic sensory area and collecting the at least one electrophysiological signal of a symptomatic sensory area comprising using electroencephalogram equipment.

17. The method of claim 1, wherein the step of collecting at least one electrophysiological signal of an asymptomatic sensory area occurs at a first time during a first experiment and the step of collecting at least one electrophysiological signal of a symptomatic sensory area occurs at a second time during a second experiment, the second time being remote from the first time.

18. The method of claim 1, wherein the step of collecting at least one electrophysiological signal of a symptomatic sensory area occurs at substantially the same time as the step of collecting at least one electrophysiological signal of an asymptomatic sensory area.

19. The method of claim 1, wherein the electrophysiological signal comprises an evoked potential technique.

20. The method of claim 1, further comprising prescribing medical treatment based at least in part on the assessed physiological condition.

21. The method of claim 1, further comprising evaluating the efficacy of a substance based at least in part on the assessed physiological condition.

22. The method of claim 1, further comprising comparing the asymptomatic response data and symptomatic response data to subjective data relating to the physiological condition.

23. The method of claim 22, further comprising scaling the asymptomatic response data, symptomatic response data, and subjective data to a common scale prior to comparing the asymptomatic response data, symptomatic response data, and subjective data.

24. The method of claim 22, further comprising acquiring subjective data.

25. The method of claim 22 wherein the subjective data comprises self-reports of pain.

26. A physiological assessment method of the type useable to assess a physiological condition, the physiological assessment method comprising the steps of:
(A) measuring at least a first electrophysiological signal of asymptomatic tissue comprising the steps of:
(a) attaching a stimulus unit to an asymptomatic sensory area;
(b) stimulating the asymptomatic sensory area; and
(c) collecting at least one electrophysiological signal of the asymptomatic sensory area to obtain first asymptomatic response data;
(B) measuring at least a first electrophysiological signal of symptomatic tissue comprising the steps of:
(a) attaching a stimulus unit to a symptomatic sensory area;
(b) stimulating the symptomatic sensory area;
(c) waiting for a predetermined time period; and
(d) collecting at least one electrophysiological signal of the symptomatic sensory area to obtain first symptomatic response data.

27. The method of claim 26, further comprising:
(A) obtaining subjective data concerning a physiological condition; and
(B) comparing the asymptomatic response data, the symptomatic response data, and the subjective data to assess a physiological condition.

28. The method of claim 27, further comprising, prior to the step of comparing the asymptomatic response data, the symptomatic response data, and the subjective data, scaling the asymptomatic response data to a first scale, scaling the symptomatic response data to a second scale, and scaling the subjective data to a third scale.

29. The method of claim 28, wherein the first scale, second scale, and third scale are the same.

30. The method of claim 27, the subjective data comprising self-reported observations of the physiological condition.

31. The method of claim 30, the self-reported observations of the physiological conditions comprising self reported feelings of pain.

32. The method of claim 27, wherein the subjective data, asymptomatic response data and the symptomatic response data are acquired at substantially the same time.

33. The method of claim 26, further comprising:
(A) collecting second asymptomatic data, second symptomatic data, and second subjective data; and
(B) scaling the first and second subjective data, the first and second asymptomatic response data, and the first and second symptomatic response data by:
(a) assigning a first value to a minimum observed value of a first data element associated with the first or second symptomatic response data,
(b) assigning the first value to a minimum observed value of a second data element associated with the first or second asymptomatic response data; and
(c) assigning the first value to a minimum value of a third data element associated with the first or second subjective data.

34. The method of claim 33, the step of scaling the subjective data, the asymptomatic response data, and the symptomatic response data further comprising:
(A) assigning a second value to a maximum observed value of a fourth data element associated with the first or second symptomatic response data;
(B) assigning the second value to a maximum observed value of a fifth data element associated with the first or second asymptomatic response data; and
(C) assigning the second value to maximum value of a sixth data element associated with the first or second subjective data.

35. The method of claim 34, further comprising scaling the subjective data, the asymptomatic response data, and the symptomatic response data between the first value and the second value.

36. The method of claim 26, the step of comparing the asymptomatic response data to the symptomatic response data to assess a physiological condition comprising comparing the amplitude of an inflection point associated with the asymptomatic response data to the amplitude of an inflection point associated with the symptomatic response data.

37. The method of claim 26 wherein the physiological condition comprises a somatosensory condition.

38. The method of claim 37, wherein the somatosensory condition comprises pain.

39. The method of claim 26, wherein the step of collecting at least one electrophysiological signal of the symptomatic sensory area occurs at substantially the same time as the step of measuring the electrophysiological signal of the asymptomatic sensory area.

40. The method of claim 26, further comprising comparing the asymptomatic data to the symptomatic data to confirm the presence or absence of a physiological condition.

41. The method of claim 40, wherein the physiological condition comprises pain.

42. The method of claim 26, further comprising comparing the asymptomatic data to the symptomatic data to assess a physiological condition.

43. A method for measuring the attenuation of a somatosensory condition comprising:
(A) using an electrophysiological technique to obtain somatosensory asymptomatic response data from an asymptomatic sensory area;
(B) using an electrophysiological technique to obtain somatosensory symptomatic response data from a symptomatic sensory area;
(C) normalizing the symptomatic response data and the asymptomatic response data;
(D) normalizing subjective data; and
(E) determining the attenuation of a somatosensory condition by comparing the normalized asymptomatic data, the normalized symptomatic data, and the normalized subjective data.

44. The method of claim 43, wherein the electrophysiological technique comprises an evoked potential technique.

45. The method of claim 43, wherein the somatosensory condition comprises pain.

46. The method of claim 43, further comprising acquiring subjective response data.

47. The method of claim 46, wherein the step of acquiring at least a portion of the subjective response data occurs at substantially the same time as the step of using an electrophysiological technique to obtain somatosensory asymptomatic response data from an asymptomatic sensory area and the step of using an electrophysiological technique to obtain somatosensory symptomatic response data from a symptomatic sensory area.

48. The method of claim 46, wherein the step of acquiring subjective response data comprises filling out a self evaluation form.

49. The method claim 43, wherein the step of normalizing the symptomatic response data and the asymptomatic response data comprises:
(A) assigning a maximum value to a data element associated with the symptomatic response data and to a data element associated with the asymptomatic response data;
(B) assigning a minimum value to a data element associated with the symptomatic response data and to a data element associated with the asymptomatic response data; and
(C) normalizing remaining data elements associated with the symptomatic response data and the asymptomatic response data between the maximum value and the minimum value.

50. The method of claim 49, further comprising:
(A) assigning the maximum value to a data element associated with the subjective data;
(B) assigning the minimum value to a data element associated with the subjective data; and
(C) normalizing remaining data elements associated with the subjective data between the maximum value and the minimum value.
